(19) United States
(12) Patent Application Publication
(10) Pub. No.: US 2008/0119834 A1
(43) Pub. Date: May 22, 2008

(54) Cryosurgical System with Disposable Cryoprobe Portion

(76) Inventors: David W. Vancelette, San Diego, CA (US); Michael W.V. Perkins, Minnetonka, MN (US)

Correspondence Address: AMS RESEARCH CORPORATION, 10700 BREN ROAD WEST, MINNETONKA, MN, 55343, US

(21) Appl. No.: 11/942,038

(22) Filed: Nov. 19, 2007

Related U.S. Application Data
(60) Provisional application No. 60/866,264, filed on Nov. 17, 2006.

(51) Int. Cl. A61B 18/02

(52) U.S. Cl. 606/20

(57) ABSTRACT

A cryosurgical system and a cryoprobe having a disposable portion for use in such a system. A representative cryoprobe for use in a cryosurgical system can have a disposable portion that connects via a coupler to a non-disposable portion that is permanently attached to the cryosurgical system. A vacuum pump located in a control console of the cryosurgical system can be used to remove air that is introduced into the system when the disposable portion is connected to the non-disposable portion. Refrigerant can then be circulated through the system so that a cryothermal treatment can be performed. After the cryothermal treatment, the refrigerant can be removed from the cryoprobe and stored in a storage reservoir in the control console. Upon completion of the cryosurgical treatment, the disposable portion can then be detached and discarded.

### PRIORITY CLAIM

The present application claims priority to U.S. Provisional Application Ser. No. 60/866,264, filed Nov. 17, 2006 and entitled “CRYOSURGICAL SYSTEM WITH DISPOSABLE CRYPROBE PORTIONS”, which is herein incorporated by reference in its entirety.

### FIELD OF THE INVENTION

The present disclosure relates to cryosurgical systems for treatment of benign or cancerous tissues. In particular, the present disclosure relates to a closed loop cryosurgical system that utilizes cryoprobes having disposable portions.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used to treat prostate cancer and benign prostate disease. Cryosurgery also has gynecological applications. In addition, cryosurgery may be used for the treatment of a number of other diseases and conditions including, but certainly not limited to, breast cancer, liver cancer, renal cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

### SUMMARY OF THE INVENTION

The present disclosure is directed to a cryosurgical system and a cryoprobe having a disposable portion for use in such a system. A representative cryoprobe for use in a cryosurgical system can have a disposable portion that connects via a coupler to a non-disposable portion that is permanently attached to the cryosurgical system. A vacuum pump located in a control console of the cryosurgical system can be used to remove air that is introduced into the system when the disposable portion is connected to the non-disposable portion. Refrigerant can then be circulated through the system so that a cryothermal treatment can be performed. After the cryothermal treatment, the refrigerant can be removed from the cryoprobe and stored in a storage reservoir in the control console. Upon completion of the cryosurgical treatment, the disposable portion can then be detached and discarded.

In one aspect of the present disclosure, a cryoprobe has a disposable portion and a non-disposable portion. The disposable portion and non-disposable portion can each include one half of a coupler with which they can connect together. When assembled, the coupler can include fluid channels linking fluid inlets and outlets between the non-disposable portion and the disposable portion and can further include one or more check valves to control the fluid flow. The disposable portion can comprise a capillary tube or other suitable Joule-Thomson expansion element to expand refrigerant in order to form ice balls on a conductive freeze tip for performing a cryothermal treatment. Both disposable portion and non-disposable portion can include insulation to reduce heat transfer between refrigerant in the fluid channels and the body/ambient air.

In another aspect of the present disclosure, a cryosurgical system can utilize cryoprobes having disposable portions. The cryosurgical system can include a control console that directs refrigerant from one or more compressors into one or more cryoprobes and receives returned refrigerant from a conductive freeze tip through a flexible line. Cryoprobes can each include a generally permanent, non-disposable portion attached to the cryosurgical system that can be connected to various disposable portions usable for individual cryosurgical applications. The control console can further include a storage reservoir for retaining refrigerant while the disposable portions are attached and detached and a vacuum pump that can be used to evacuate fluids such as air and refrigerant from the system.

In yet another aspect of the present disclosure, a method of performing a cryosurgical procedure can use cryoprobes having disposable portions. A new, sterile disposable portion can first be connected to a non-disposable portion of a cryoprobe with a coupler. Initially, the fluid inlets and outlets of the disposable portion can be cut off from the system by check valves in the coupler. A vacuum pump in a control console can then be used to remove the air introduced into the system from attaching the disposable portion. The check valves can then be opened and refrigerant allowed to flow through the system. The refrigerant flows through the cryoprobe resulting in the formation of ice balls on a conductive freeze tip of the disposable portion and a cryothermal treatment can be performed. After the cryothermal treatment, the refrigerant in the system can be returned to the compressor and can be stored within a storage reservoir in the control console. The disposable portion can then be detached and discarded, while a new, sterile disposable portion can be attached to the non-disposable portion to begin a new, subsequent cryosurgical procedure.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a side view of an embodiment of a cryosurgical system according to the present disclosure.

FIG. 2 is a partial, section view of an embodiment of a cryoprobe according to the present disclosure.

FIG. 3 is a schematic view of an embodiment of a cryosurgical system for use with the cryoprobe of FIG. 2.

### DETAILED DESCRIPTION

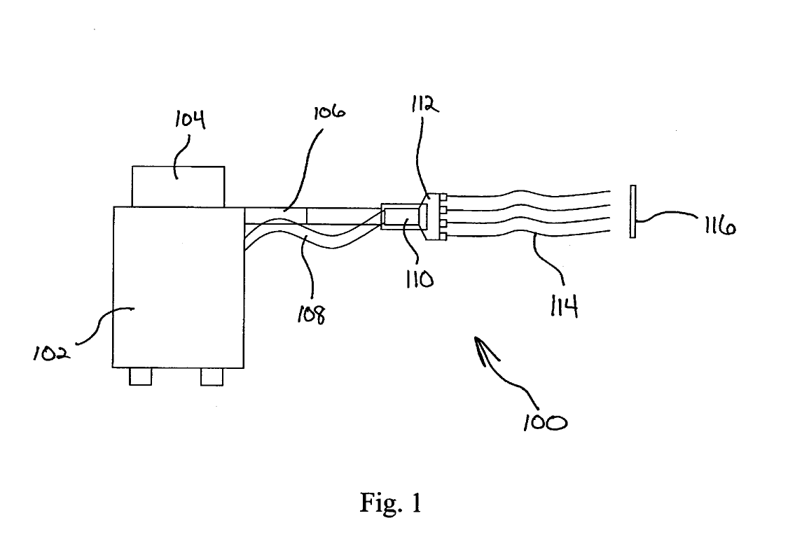

A closed loop cryosurgical system 100 according to the present disclosure is illustrated generally in FIG. 1. Cryosurgical system 100 can include a refrigeration and control console 102 with an attached display 104. Control console 102 can contain a primary compressor to provide a primary pressurized, mixed gas refrigerant to the system and a secondary compressor to provide a secondary pressurized, mixed gas refrigerant to the system. The use of mixed gas refrigerants is generally known in the art to provide a dramatic increase in cooling performance over the use of a single gas refrigerant. Control console 102 can also include controls that allow for the activation, deactivation, and modification of various system parameters, such as, for example, gas flow rates, pressures, and temperatures of the mixed gas refrigerants. Display 104 can provide the operator the ability to monitor, and in some embodiments adjust, the system to ensure it is performing properly and can provide real-time display as well as recording and historical displays of system parameters. One exemplary console that can be used with an embodiment of the present invention is used as part of the Her Option® Office Cryoablation Therapy available from American Medical Systems of Minnetonka, Minn.

With reference to FIG. 1, the refrigerant is transferred from control console 102 to a cryostat heat exchanger module 110 through a flexible line 108. The cryostat heat exchanger module 110 can include a manifold portion 112 that transfers refrigerant into and receives refrigerant out of one or more cryoprobes 114. The cryostat heat exchanger module 110 and cryoprobes 114 can also be connected to the control console 102 by way of an articulating arm 106, which can be manually or automatically used to position the cryostat heat exchanger module 110 and cryoprobes 114. Although depicted as having the flexible line 108 as a separate component from the articulating arm 106, cryosurgical system 100 can incorporate the flexible line 108 within the articulating arm 106. A positioning grid 116 can be used to properly align and position the cryoprobes 114 for patient insertion.

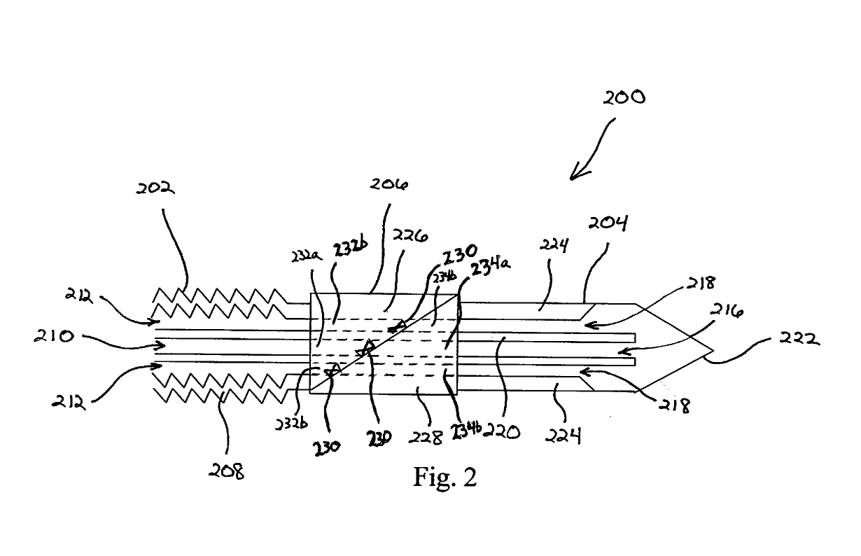

A representative cryoprobe 200 according to the present disclosure is depicted in FIG. 2. Cryoprobe 200 can comprise a non-disposable base portion 202 and a disposable end portion 204 that can connect to one another with a coupler 206. Non-disposable base portion 202 can include one or more fluid inlets 210 and one or more fluid outlets 212. Insulation 208 can surround non-disposable base portion 202 to reduce and substantially eliminate heat transfer between the refrigerant within the cryoprobe 200 and the surrounding body/ambient air. Insulation 208 can comprise any suitable insulation, such as, for example, vacuum insulation, aerogel, or foam.

Disposable end portion 204 can also include one or more fluid inlets 216 and outlets 218. Fluid inlet 216 can include a capillary tube 220 or other suitable Joule-Thompson expansion element. Refrigerant can flow through capillary tube 220 and be isenthalpically expanded to further reduce the refrigerant's temperature such that an ice ball is formed on a conductive freeze tip 222, which is subsequently used to perform a cryothermal treatment. In some representative embodiments, conductive freeze tip 222 can resemble a trocar configuration so as to aid in tissue penetration such as during a prostate treatment procedure.

Disposable end portion 204 can include insulation 224 to prevent heat transfer from the body or ambient air to the refrigerant as it returns along fluid outlets 218. Insulation 224 can comprise any suitable insulation, such as, for example, vacuum insulation, aerogel, or foam. Where cryoprobe 200 is vacuum insulated, vacuum insulation 224 in disposable end portion 204 can be separately sealed from vacuum insulation 208 in non-disposable base portion 202.

Coupler 206 can comprise a two part assembly including a first coupling member 226 and a second coupling member 228. In one presently preferred embodiment, the coupler 206 comprises a quick-connect style coupling allowing the first coupling member 226 and second coupling member 228 to be quickly and easily joined. First coupling member 226 and second coupling member 228 can be joined using compression or threaded joining techniques. First coupling member 226 can be integral to the non-disposable base portion 202 while the second coupling member 228 can be integral to the disposable end portion 204. To connect disposable end portion 204 with non-disposable base portion 202, second coupling member 228 is attached to first coupling member 226. When coupler 206 is operably joined, fluid inlets 210, 216 and fluid outlets 212, 218 are sealingly, fluidly connected.

Referring again to FIG. 2, coupler 206 can use various seal types and methods, including o-rings, elastomers, and metals seals, to sealingly isolate fluid inlets 210, 216 and fluid outlets 212, 218. First coupling member 226 can include fluid inlet fluid channel 232a and fluid outlet channels 232b and second coupling member 228 can fluid inlet channel 234a and fluid outlet channels 234b. When first coupling member 226 and second coupling member 228 are operably connected, fluid inlet channel 232a and fluid inlet channel 234a fluidly interconnect fluid inlet 210 with fluid inlet 216 while fluid outlet channels 232b and fluid outlet channels 234b fluidly interconnect fluid outlets 212 with fluid outlets 218. First coupling member 226 can include check valves 230 within fluid inlet channel 232a and fluid outlet channels 232b. Check valves 230 can comprise normally closed valves that can generally isolate fluid inlet 210 and fluid outlets 212 when first coupling member 226 is disconnected from second coupling member 228 to prevent air from entering or refrigerant fluid from escaping the system. When second coupling member 228 is attached to first coupling member 226, check valves 230 can be forced open, providing for a free flow of refrigerant through fluid channels 232. Upon subsequently detaching the second coupling member 228, check valves 230 can close and again seal off the system. In one representative embodiment, check valves 230 can comprise spring biased check valves.

The fluid inlets 210, 216 and fluid outlets 212, 218 can be arranged in coaxial or side by side relation. Insulation can also be provided to coupler 206 to reduce and/or substantially eliminate heat transfer between the refrigerant and the surrounding body/ambient air.

Referring again to FIG. 2, coupler 206 can also include spring-biased check valves 230 that can be used to selectively control refrigerant flow. Valves 230 can be located within each fluid channel 232 in first coupling member 226. Valves 230 can be biased such that they are in a closed position preventing air from entering or fluid from escaping the system when first coupling member 226 is not engaged with second coupling member 228. When second coupling member 228 is attached to first coupling member 226, the spring-biased valves 230 can be forced open, providing for a free flow of refrigerant through fluid channels 232. Upon subsequently detaching the second coupling member 228, the valves snap back shut, again sealing off the system.

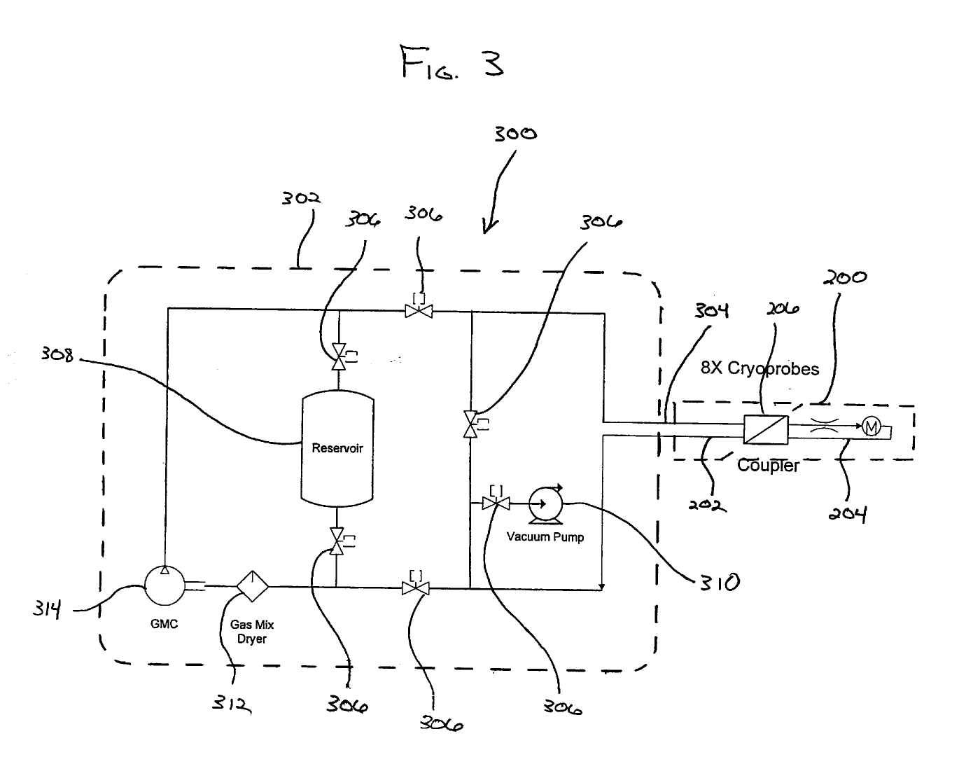

A representative closed-loop cryosurgical system 300 for use with one or more of cryoprobe 200 is illustrated schematically in FIG. 3. Cryosurgical system 300 can include a control console 302 and a flexible line 304 fluidly connected to cryoprobe 200. Though not depicted, cryosurgical system 300 can make use of display 104 for displaying information and/or controlling operating characteristics of the cryosurgical system 300. A plurality of flow valves 306 are used throughout cryosurgical system 300 as to route and/or purge refrigerant based on the operating mode of the cryosurgical system 300. Control console 302 can include a storage reservoir 308, a vacuum pump 310, gas mix dryer 312 and a gas mix compressor 314 to provide a primary pressurized, mixed gas refrigerant to the system. In some embodiments, cryosurgical system 300 can include additional components such as, for example, a secondary compressor to provide a secondary pressurized, mixed gas refrigerant to the system, a refrigerant buffer tank to make up small of amounts of refrigerant lost during attachment and detachment of disposable end portion 204 and non-disposable base portion 202, a refrigerant pre-cooler and the like.

To use cryosurgical system 300 according to the present disclosure, a new, sterile disposable cryoprobe portion 204 can first be attached to the non-disposable cryoprobe portion 202. Prior to connection, check valves 230 isolate fluid inlet 210 and fluid outlets 212 from the ambient environment. Once the coupler 206 is connected, check valves 230 are forced to an open position wherein vacuum pump 310 can be used to evacuate any air that was introduced into the cryosurgical system 300 during connection of the disposable cryoprobe portion 204 and non-disposable cryoprobe portion 206 or any air previously introduced when a prior non-disposable cryoprobe portion 206 was discarded. With check valves 230 in the open disposition, refrigerant can be pumped from storage reservoir 308 back into the full circuit through all of the fluid inlets 210, 216 and outlets 212, 218. Once the conductive freeze tip 222 has been sufficiently cooled, a cryothermal treatment can be performed.

Once the cryothermal treatment has been completed, the refrigerant that is being pumped from gas mix compressor 314 into the cryoprobes 200 can be redirected into the storage reservoir 308. The refrigerant remaining in the fluid inlets 210, 216 and outlets 212, 218 can also pumped back into the storage reservoir 308. The pressure within the disposable end portion 204 may be below atmospheric pressure and can be vented. Alternatively, dry nitrogen can be introduced to purge the system. Disposable end portion 204 can then be removed and a new, sterile disposable end portion 204 can be attached to non-disposable base portion 202 such that cryosurgical system 300 can be used to perform another cryosurgical procedure.

Disposable cryoprobe 200 provides for a sterile instrument without the need for a separate sheath or cover. This allows for smaller and more efficient cryoprobes. Use of a detachable cryoprobe 100 also provides a more versatile cryosurgical system 200 because variously configured cryoprobes for different applications can be easily attached and detached. For instance, disposable end portion 204 can be selectively sized and shaped to correspond with designated types and areas of cryosurgical treatment. In some representative embodiments, disposable end portion 204 can comprise a lengthened flexible portion that operably connects to the non-disposable base portion 202.

To increase the length of the sterile barrier, a disposable drape or sheath can be used to cover non-disposable base portion 202 during cryothermal treatment. Drape can be a separate component of the system or can attach and unfurl from disposable portion 202. After each cryosurgical procedure, drape can be separately discarded or will be discarded along with disposable portion 202.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A cryoprobe for use in cryosurgical procedure, comprising:
a non-disposable base portion including one or more base fluid inlets and one or more base fluid outlets;
a disposable end portion including one or more end fluid inlets and one or more end fluid outlets and a conductive freeze tip configured to perform a cryosurgical procedure; and
a coupler configured to connect the non-disposable base portion and the disposable end portion, the coupler comprising a first coupling member integrally formed with the non-disposable base portion and a second coupling member integrally formed with the disposable end portion, the first coupling member including at least one first coupling inlet channel and at least one first coupling outlet channel, the second coupling member including at least one second coupling inlet channel and at least one second coupling outlet channel, such that connection of the first coupling member and the second coupling member fluidly interconnect the base fluid inlets with the end fluid inlets and the base fluid outlets with the end fluid outlets.

2. The cryoprobe of claim 1, wherein the first coupling member includes a check valve within each first coupling inlet channel and each first coupling outlet channel.

3. The cryoprobe of claim 1, wherein at least one portion of the cryoprobe is surrounded by insulation, the at least one portion of the cryoprobe selected from the group consisting of the non-disposable base portion and the disposable end portion.

4. The cryoprobe of claim 3, wherein the insulation is selected from the group consisting of: vacuum insulation, aerogel and foam.

5. The cryoprobe of claim 1, wherein the one or more end fluid inlets includes a Joule-Thompson expansion element.

6. The cryoprobe of claim 1, wherein the conductive freeze tip has a trocar configuration.

7. A closed loop cryosurgical system comprising:
a console containing a refrigerant;
one or more cryoprobes fluidly connected to the console, each cryoprobe including a non-disposable base portion, a disposable end portion and a coupler configured to connect the non-disposable base portion and the disposable end portion, the coupler comprising a first coupling member integrally formed with the non-disposable base portion and a second coupling member integrally formed with the disposable end portion; and
a vacuum pump contained within the console, the vacuum pump configured to remove air from each cryoprobe introduced when disposable end portion is connected to non-disposable base portion.

8. The system of claim 7, further comprising:
one or more valves configured to selectively control the flow of the refrigerant to the one or more cryoprobes.

9. The system of claim 7, wherein the first coupling member includes a normally closed check valve in at least one first coupling inlet channel and in at least one first coupling outlet channel.

10. The system of claim 7, wherein each cryoprobe includes a tip portion having a trocar configuration.

11. The system of claim 7, wherein each disposable end portion includes a Joule-Thompson expansion element in a disposable fluid inlet channel.

12. A method of performing a cryosurgical procedure, comprising:
attaching a disposable end portion of a cryoprobe to a non-disposable base portion of the cryoprobe;
activating a vacuum pump located in a control console to remove air introduced when disposable end portion is attached to non-disposable base portion;
circulating a refrigerant contained in a storage reservoir in the control console through the cryoprobe to cool a conductive freeze tip of disposable end portion; and
performing a cryothermal treatment with the conductive freeze tip.

13. The method of claim 12, further comprising:
returning the refrigerant to the storage reservoir.

14. The method of claim 12, wherein attaching the disposable end portion to the non-disposable base portion opens at least one check valve in a non-disposable fluid inlet and at least check valve in a non-disposable fluid outlet.

15. The method of claim 14, further comprising:
detaching the disposable end portion from the non-disposable base portion wherein the at least one check valve in the non-disposable fluid inlet and the at least one check valve in non-disposable fluid out are closed.

16. The method of claim 15, further comprising:
disposing of the disposable end portion.

17. The method of claim 12, wherein the disposable end portion attaches to the non-disposable base portion with a coupler, the coupler comprising a first coupling member integrally formed with the non-disposable base portion and a second coupling member integrally formed with the disposable end portion.
